(19) United States
(12) Patent Application Publication
(10) Pub. No.: US 2022/0036987 A1
(43) Pub. Date: Feb. 3, 2022

(54) UTILIZING HEALTHCARE PROVIDERS NETWORK EFFECT TO INCREASE COMPLIANCE FOR BETTER HEALTH OUTCOMES

(71) Applicant: PREDICTMEDIX INC., Toronto (CA)

(72) Inventors: Rajiv Muradia, Ottawa (CA); Rahul Kushwah, Toronto (CA)

(21) Appl. No.: 17/385,889

(22) Filed: Jul. 26, 2021

Related U.S. Application Data
(60) Provisional application No. 63/058,567, filed on Jul. 30, 2020.

(51) Int. Cl. G16H 20/00; G06N 20/00; G16H 50/70

(52) U.S. Cl. CPC G16H 20/00; G16H 50/70; G06N 20/00

(57) ABSTRACT

The novel invention provides for a system and a method to motivate care providers to ensure (a) compliance for a prescribed medical protocol for maintaining good health, and (b) collectively work together for speedy recovery of patients. The disclosed method and system help the care providers and care partner network to follow the prescribed medical protocols. The care providers are given the task of improving the health of patients. For the efforts, the care providers are rewarded with reward points based on improved health condition of patients. The rewards are recorded in the database as reward points. The reward points are converted into normalized points. These normalized points are credited into the account of the care providers as a reward for compliance with the treatment protocols.

### RELATED APPLICATIONS

This application is related to, and claims priority to, the following:
1. Provisional Application Ser. No. 63/048,152, filed Jul. 5, 2020.
2. Provisional Application Ser. No. 63/048,131, filed Jul. 5, 2020.
3. Provisional Application Ser. No. 63/058,567, filed Jul. 30, 2020.

The subject matter of the related applications, each in its entirety, is expressly incorporated herein.

### FIELD OF THE INVENTION

The present invention provides a novel method and system of management of rewards for care provider network for assisting in improved health of the patient and for compliance to a treatment protocol. More specifically, the invention disclosed a partner reward management system and method for care partners/care providers to collectively contribute to improved health of the patient and accordingly rewarding the care providers and other wellness partners.

### BACKGROUND OF THE INVENTION

Due to large scale outbreak of various infectious, non-infectious, heredity and other lifestyle diseases, it has become important to ensure that patients strictly follow the treatment protocol to live a healthy life. The patient might procrastinate the schedule of medication, exercise and diet, which has been set by the medical expert. This results in deterioration of health of the patient leading to further complications. For example, the pre-diabetic person can be treated by lifestyle management by following a prescribed protocol of diet, exercise, and medicine. Most of the people are not motivated to strictly follow the prescribed diet, exercise and medicine plan resulting in aggravation of the disease.

Likewise, a heart patient can manage good health by following a treatment protocol of diet and exercise coupled with medications. By following a strict diet, exercise and medicine plan, the heart patient can reduce the chances of the heart attack. Therefore, it is important to motivate the patient to strictly follow the prescribed protocol by a medical expert to maintain good health. Furthermore, it is equally important to motivate the care providers to monitor such compliance by providing an incentive scheme.

The novel invention provides for a system and a method to motivate care providers and/or wellness providers to ensure (a) compliance of a prescribed medical protocol for maintaining good health, and (b) collectivily working together for a speedy recovery of the patient. The disclosed method and system help the care providers/wellness providers/care partners and care partner network to follow the prescribed medical protocol. The care providers/wellness partners/care providers are given the task of improving the health of the patient. For the efforts, the care provider/wellness partner/care partner are rewarded with reward points based on improved health condition of patients. The rewards are recorded in the database as reward points. The reward points are converted into normalized points. These normalized. points are credited into the account of the care provider/wellness partner/care partner as a reward for adhering to compliance of the treatment protocol.

### SUMMARY OF THE INVENTION

Methods and systems are disclosed for a healthcare compliance framework with a two-sided marketplace. The first side of the marketplace are patients having one or more care providers to assist with their health where the patient is ailing with at least one disease. The care provider's primary responsibility is to ensure good health for their patients. The other side of the marketplace is care providers that provide services to the patients for keeping them healthy. A partner reward management system comprising patients and care providers is disclosed. The partner reward management system utilizes at least two key metrics, “On Track” and “On Target” in addition to other ancillary metrics such as Task Duration, Patient Satisfaction Surveys, Patient Disease Awareness Surveys to determine health outcomes for earning rewards. The “On Track” metric measures patient's compliance in following their daily care plan regimens, the “On Target” metric measures patient's success in keeping their vital sign measurements within the acceptable ranges as determined by their care providers. The “Task Duration” measures the time a specific care provider/care partner spends with patient as their health condition gets better. The “Patient satisfaction Survey” measures patient's satisfaction based on their condition, tasks being performed, care providers/care partners engaged and their rating. The “Patient Disease Awareness Survey” measures patient's understanding of their disease condition, tasks being performed as well as their understanding of their care plan regimens.

In embodiments, the partner reward management method and system may include a care partner network. The care partner network comprises care management partners, wellness management partners, care providers, nurses, doctors, pharmacy, hospitals, and healthcare volunteers.

In embodiments, the care partner network team are assigned tasks. The tasks can be assigned to individuals or assigned collectively to a care provider team depending on the health condition of the patient. The tasks have different weightage options for earning rewards based on the health condition of the patient and their contribution and final outcome. For example, a patient with chronic depression may benefit more from regular visits by a personal support worker to go for a walk, just having a conversation or other interactions with the patient as compared to getting medications from a nurse to manage the patient's condition.

In embodiments, the partner reward management system may enable various healthcare providers such as doctors, specialists, family members, informal care team (including neighbors, friends etc.) and healthcare workers/volunteers in the care of the patient's health outcome to contribute in a holistic improvement of the patient health, thereby earning rewards for themselves.

In some embodiments, the health care provider may be associated with multiple patients thereby considerably increasing the income from rewards.

The partner reward management method and system allow every patient to receive treatment and manage his/her health. Initially, the partner reward management method and system evaluates the health condition of each patient. Depending on their health condition, the partner reward management method and system provides an initial wellness index number. In some embodiment, the wellness index may define the number of care providers required by the patient. Based on how many care providers are involved with the patient's care, they can be eligible to participate in earning reward.

In embodiment, the reward may be in inform of care points. In other embodiments, the awards may be in form of monetary or non-monetary benefits.

In embodiments, the care management partners may include different care professionals to be included in the patient's care network.

In embodiments, the different care professionals may include wellness partners, doctors, care providers, nurses, health care volunteers and other type of medical workers.

The care management partners team may involve each other in the care of patient's health and improve outcome. As a reward, the care management partners are awarded points with each measured outcome metrics. The two major metrics, which are evaluated, are on Task and On Target. In addition, other metrics that may be evaluated include (a) Task Duration, (b) Patient Satisfaction Surveys, (c) Patient Disease Awareness Surveys, (d) Care Partner Feedback and (e) collaborative Assessment of care partner management to reach desired goal for achieving the on Task and on Target metrics.

In some embodiments, each of the care management partners will create a collaborative plan to engage with each other to participate in patient's care while being rewarded. In some embodiments, the rewards may be proportional to the contribution of each of the care management partners.

In some embodiments, the partner reward management module associated with the partner reward management method and system may automatically allocate different health care providers/professionals and based on the contribution of each of the health care providers/professionals, automatically provide allocation of rewards to each of the care providers/professionals. Furthermore, the partner reward management system may provide an implementation of learning algorithms to automatically reward each of the health care professional.

In embodiments, the partner reward management module associated with the partner reward management method and system may facilitate and encourage the health care network to add other health care partners for improving the health of the patient. The patient's care network may grow by enabling the patient to have additional improved outcome. Thus, the partner reward management module provides improved health condition and simultaneously rewards each health care partner for their contribution in improving the health of the patient.

In embodiments, the accumulated points and received rewards from the partner reward management module by one or snore health care professionals may be shared or transferred to, or redeemed for, associated partners, for example pharmacies, food joints, retail stores etc.

In some embodiments, the care network providers may have various roles including share educational content, assist with social factors, motivational reach out, “nudging”, clinical focus, regular visitations to improve patient well being state, etc.

The reward for improvement of health of the patient is intended to motivate the care network team/care partner network. Further, the care network team/care partner network receives rewards for influencing and changing the behavior of the patient for improved health condition.

In embodiments, the accumulation and the quantity of points may be based on the short term and the long term improvement of patient's health.

The partner reward management method and system may implement an algorithm in a machine learning module; the machine learning module comprising an analytical database, a rule based engine, a decision tree, a recommendation engine; the algorithm in the machine learning module enables a clinical administrator to set up priorities of the disease conditions being managed for one patient and/or a number of patients with a specific disease. In addition, the partner reward management system and method also create the disease management and prescription protocol for management of the disease. Further, the the partner reward management method and system may allocate the required number of care management partners and may also create the list of tasks, targets and improvement index for each patient separately. In some embodiments, the partner reward management method and system may create sub-tasks for main tasks or independently create additional tasks such as (a) Task Duration, (b) Patient Satisfaction Surveys, (c) Patient Disease Awareness Surveys, (d) Care Partner Feedback and (e) Collaborative Assessment of care partner management to reach desired goal for achieving the on Task and the on Target metrics. Accordingly, the rewards points are allocated as a pool to care provider network towards each condition.

The partner reward management method and system may further implement other algorithms that assess the contribution of each task performed by the care network team and the metrics generated by the patient related to the current condition and improvement index. In sonic embodiments, the improvement index may be related to the progress of the patient.

When the tasks related to the patient fail to improve the metrics related to patient's health or alternatively the health of the patient degrades then the network member associated with the tasks may have their points adjusted to reflect this contribution, where the adjustment may be addition or removal of points from the pool. The deep learning neural network algorithms accomplish and implement rules and accordingly provide instruction to add or reduce points based on the outcome of the patient's health.

### DESCRIPTION OF FIGURES

Different embodiments will now be described in detail with reference to the drawings, in which:

FIG. 1 illustrates a block diagram of the environment in which a partner reward management system operates in accordance with an embodiment of the present invention;

FIG. 2 illustrates different components of a partner reward management module of the partner management system in accordance with an embodiment of the present invention;

FIG. 2A illustrates different components of a disease module of the partner reward management module in an embodiment of the present invention;

FIG. 2B illustrates different components of a care module of the partner reward management module in an embodiment of the present invention;

FIG. 3 illustrates a partner reward management system in accordance with an embodiment of the present invention;

FIG. 4 illustrates a process flowchart of care management system in accordance with an embodiment of the invention;

### DETAILED DESCRIPTION OF THE INVENTION

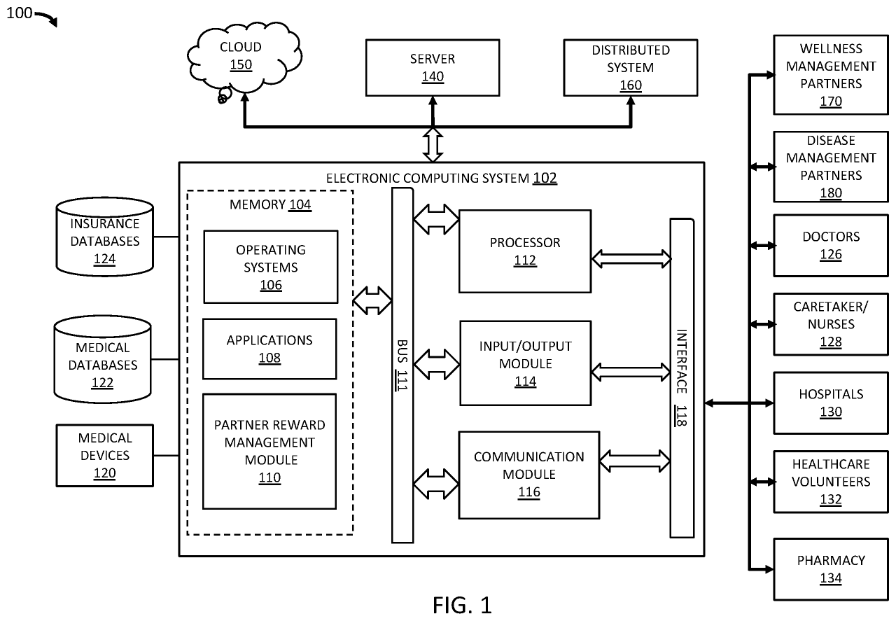

FIG. 1 illustrated the overall environment of a partner rewards management system. The overall environment 100 comprises an electronic computing system/device 102 networked with a cloud 150, a server 140, a distributed system 160, an insurance database 124, a medical database 122, and medical device(s) 120. The overall environment 100 for the reward management system further includes one or more wellness management partners 170, one or more disease management partners 180, one or more doctors 126, one or more caretakers 128, one or more health care volunteers 132, one or more hospitals 130, and one or more pharmacy 134.

The electronic computing system/device 102 includes a memory 104 comprising an operating system 106, one or more applications 108, and a partner reward management module 110 interfaced with an internal bus 111 to a processor 112, one or more input/output devices 114, and one or more communication devices 116. The interface bus carries data and supplies electrical current among different modules. In addition, the electronic computing system/device 102 includes an interface 118, which allows the electronic computing device 102 to connect with the server 140, the medical database 122 and the cloud 150. Furthermore, the external entities such as the server 140, the medical database 122 and the cloud 150 can be accessed by the electronic computing system/device 102 through an interface 118. The internal modules and entities such as processor 112, communication module 116, or the partner reward management module 110 in memory 104 are interconnected and accessible to each other through the internal bus 111 and access and be accessible to the external entities through the interface 118.

The patient may own or control the electronic computing device 102 for managing and tracking his/her own health.

In some embodiments, the electronic computing device 102 may be desktop computer owned by the customer and connected with the Internet.

In some embodiments, the electronic computing device 102 may be a mobile device or a medical device with mobile connectivity, which is owned by the customer and connected with the Internet.

The medical devices 120, the one or more wellness management partners 170, the one or more care providers/nurses 128, the one or more health care volunteers 132, the one or more hospitals 130, and the one or more pharmacy 134 may be connected with the electronic computing device 102 of the patient via wired or wireless network, for example, the medical device 120 can be connected with the electronic computing device 102 by an USB interface or a LAN connection using different type of ports such as micro USB, C type and other type of interface port. In some embodiments, the port may be a physical port or a virtual port. In other embodiments, the medical devices 120 may interface via wireless connection with the electronic computing device 102 such as Bluetooth, Edge or Wi-Fi.

The partner reward management system includes the partner reward management module 110 which may be implemented into a single device or may be implemented over a distributed network. Although, the partner reward management module 110 is shown to reside in the memory 104 of the computing device 102, in other embodiments, the partner reward management module 110 may reside in part in different devices in a networked environment such as the cloud 150, the server 140, and the distributed system 160.

In some embodiments, the insurance database 124 and/or the medical database 122 associated with the partner reward management module 110 may reside on the cloud 150 or the server 140 and/or implemented as distributed system 160.

In some embodiments, the one or more doctors 126 may provide care management template or advise to care or wellness management partners 170, the care providers/nurses 128 and the healthcare volunteers 132.

In some embodiments, the one or more pharmacy 134 may provide medicine and equipment to the one or more care/wellness management partners 170, the care providers/nurses 128 and the healthcare volunteers 132.

The partner rewards management system is directed to improve the health of the patient by ensuring compliance of the prescribed protocol recommended by the medical expert. In addition, the objective of the overall environment 100 may be to monitor and ensure accomplishment of health tasks to comply with the target set for a specified period such as a day, a week, a month, a few days or a few weeks, etc.

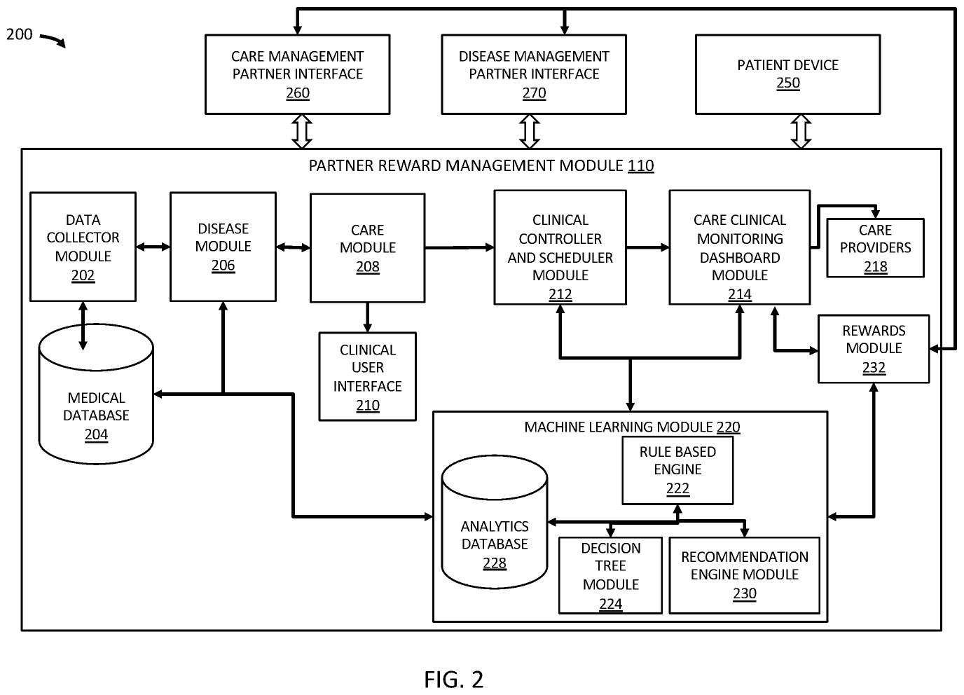

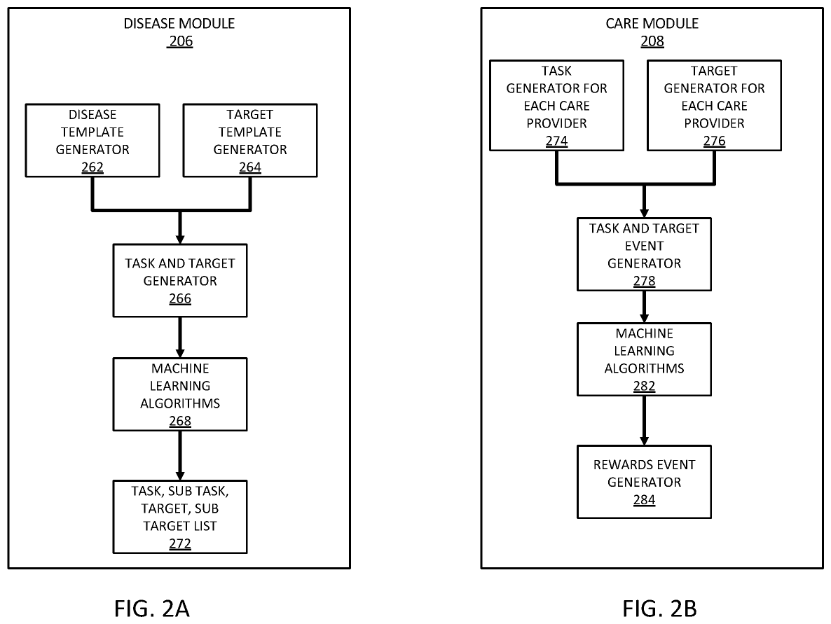

FIG. 2 illustrates different components of the partner reward management module residing inside the partner rewards management system in an embodiment of the present invention. The partner reward management module 110 allows the different stakeholder to manage and ensure compliance of the prescribed medical protocol of the patient prescribed by a medical or clinical expert, for example, a doctor.

The partner reward management module 110 comprises a data collector module 202, a medical database 204, a disease module 206, a care module 208, a clinical user interface 210, a clinical controller and scheduler module 212, a care clinical monitoring dashboard module 214, one or more care providers 218, a reward module 232 and a machine learning module 220. In addition, the partner reward management module 110 may be connected with a patient device 250, a disease management partner interface 270 and a care management partner interface 260. In the embodiment of FIG. 2, the machine learning module 220 comprises a rule based engine 222, analytic database 228, decision tree module 224 and a recommendation engine module 230.

In different embodiments, the partner reward management module 110 can be implemented in different configurations. The partner reward management module 110 can reside on the server 140 or the cloud 150 or may be distributed across the network with different components distributed over different servers and or cloud to form a distributed network.

The partner reward management system 110 comprises the data collector 202, which may collect medical data having different medical parameters. Each medical parameter may contribute directly or indirectly to prescribed treatment. The medical data/information of the patient may be aggregated using one or more medical devices 120 and/or the patient device 250.

The medical database 204 stores the medical data gathered by the data collector module 202. For example, all medical information/data related to patient may be stored in the medical database 204. In some embodiments, the medical data/information may be aggregated from other sources connected directly or indirectly to the partner reward management module 110. For example, the medical data/information may be aggregated from different sources, such as, medical history databases of the patients, family physician, hospital of the patient, clinical laboratory of the patient, government medical compliance offices, and insurance repositories/databases.

In some embodiments, the data collector module 202 captures the medical data from the patient for the medical device(s) 120 either remotely or locally using either a wired or a wireless connection. The medical data/information aggregated at the data collector module 202 may be filtered, processed and transformed to extract relevant information that is related to patient's ailment and/or diseases. Further, the patient data stored in the medical database 204 may provide insight to the medical expert for developing a customized treatment protocol by analyzing all the aliments of the patient. In some embodiments, the medical data may be entered manually into the data collector module 202 or obtained and uploaded automatically by a medical sensor using wireless technologies such as Wi-Fi, Bluetooth, Edge or others. In this embodiment, the data collector module 202 can be operated by the patient or by a health-care worker on behalf of the patient.

In some embodiments, the medical database 204 may reside on the server 140 or the cloud 150 or the distributed system 160.

In some embodiments, the medical information/data stored in the medical database 204 may include all hereditary and genetic information and life history of the patient with respect to the medical data comprising medical parameters during the patient's life span. The data may be stored in the medical database 204 persistently and the data may be structured such that each record is uniquely identifiable. In embodiments, the database may include SQL databases such as MySQL, NoSQL databases or MongoHQ, or key-value stores and file system storage such as a syslog buffer.

In some embodiments, when the partner reward management module 110 is implemented as software, it may be implemented on a server, a personal computer, a mobile smart computer or a web application executed on an electronic computing device, for example, the electronic computing device 102.

The partner reward management system 110 further includes the disease module 206 for a medical expert or a disease management partner 180 to manage the disease(s) or the aliment(s) of the patient. The medical expert or a panel of expert or the disease management partner 180 is assigned a responsibility to look into one or more aliments of the patient and accordingly suggest a template for disease management. The disease management template may include creating a schedule or template for care management such as but not limited to medication plan, exercise plan, diet plan, clinical test plan, control parameter targets, compliance plan, emergency plan in case of medical emergency, scheduled visit to hospitals, taking readings such as blood pressure, sugar level and body temperature, etc.

In some embodiments, the disease module 206 may reward one or more medical expert for preparing a plan that follows the target value of improvement per unit time, for example each day, each week, each month and the like.

In some embodiments, the disease module 206 may allow a group of medical experts to formulate a disease template, when the patient is ailing with multiples disease. The group of experts may be rewarded on proportionate basis based on the contribution of each medical expert for suggesting a treatment plan that allows a patient to recover in a predecided duration of time.

The disease module 206 provides the disease template related to patient disease management to the care module 208. The care module 208 is connected with a clinical user interface 210, which allows the clinical expert to assign tasks to one or more care providers.

Referring to FIG. 2A, the disease module comprises a disease template generator 262, a target template generator 264, a task and target generator 266, and a machine learning algorithms module 268 and task, sub-task, target and sub-target list 272. The one or more medical experts may create a disease template using disease template generator 262. Additionally, medical experts may also create a list of targets to be achieved by the patient using the target template generator 264. In embodiments, the disease template may include a list of tasks to be performed by different stakeholder such as patient, care partners, wellness partners, doctors and nurses. The tasks and targets outlined in the disease template are merged logically using the task and target generator 266. In some embodiments, the tasks and targets merging may involve rearranging the task, aligning the tasks with practical targets, redefining the tasks and the targets etc. In certain embodiments, the task and target generator 266 results may be passed to a machine learning algorithm to further modify the results based on machine learning techniques before passing the results to the module task, sub-task, target, sub-target lists 272.

In some embodiments, task and target generator 266 passed to machine learning algorithm may automatically create the task, sub-task, target, sub-target lists 272. The task, sub-task, target, sub-target lists 272 is passed to the care module 208. In some embodiments, the task, sub-task, target, sub-target lists 272 may also be passed to the clinical user interface 210.

Referring to FIG. 2B, the care module 208 includes a task generator 274 for assigning task to each care provider/care partner/wellness provider. The task generator 274 assigns tasks to each care provider/care partner or other care management partners. The care module 208 also includes target generator 276 for each care provider/care partner/wellness provider. Each task or a group of tasks may be associated with a target to achieve disease improvement or healing, meeting of those targets may result in reward for the care provider/care partner/wellness provider. The task and target event generator 278 aligns the one or more tasks with a target to be achieved. The task and target event generator 278 results may be passed on to a reward event generator 284.

In some embodiments, the task generator 274 for assigning task to each care provider/care partner/wellness provider and the target generation 276 for each care provider/care partner/wellness provider may include sub-tasks, lists, schedules and other generic schedule plan under each task generator or target generator.

In some embodiments, a machine learning algorithms module 282 may be utilized after training to predict the alignment and/or the association between the task event generator 274 and the target event generator 276 to create the task and target event generator 278 and the reward event generator 284.

Referring back to FIG. 2, in the exemplary embodiment, the clinical user interface 210 may be managed by the administrator, the project manager, the clinical expert responsible for care management of the patients, the care administrator, disease administrator or some other person responsible for partner reward management system 110.

The care module 208 allows the clinical user interface 210 to define and/or refine and/or modify the tasks, sub-tasks, allocate task to each care partner, care provider wellness partner and other clinical and non-clinical staff responsible for disease management.

In some embodiments, the one or more task can be assigned to one or more care partners, care providers, wellness partners and other clinical and non-clinical staff responsible for disease management.

In some embodiments, the task may be related to rewards, which may be specified for each task. The reward may be provided to the care partners, care providers, disease management partners on completion of the task. Furthermore, the task may include the assessment of the progress of the patient with respect to prescribed treatment protocol. For each milestone achieved results in a positive reinforcement toward patient's recovery, the care partners, the care providers, and the wellness management partners, who may be rewarded accordingly in predetermined proportions.

In embodiments, the rewards may be monetary or non-monetary or in form of reward points that can be converted into money value or redeemed against the goods/ services.

The care module 208 is connected with the clinical controller and scheduler module 212. As disclosed in FIG. 2B, care module 208 provides the task and target events, which are formalized and assigned to one or more care providers/care partners/wellness partners. The clinical controller and scheduler module 212 monitors the progress of the each of the tasks and the targets associated with each of the task. Additionally, the clinical controller and scheduler module 212 organizes and manages tasks. It maintains the log of completed tasks and scheduled tasks in an analytical database 228. The clinical controller and scheduler module 212 provides input to the machine learning module 220 for awarding rewards and storing them persistently for each task, each target, each patient, each care partner, each care provider, each wellness partner, each doctor and each disease management partner.

In the exemplary embodiment in FIG. 2, the machine learning module 220 comprises a rule based based engine 222, a decision tree module 224, analytics database 228 and a recommendation engine 230 and provides automatic management of on tasks and on target rewards. In addition, the rule based engine 222 may also manage sub-tasks, schedules, treatment plans and other templates at granular level associated with the tasks and/or targets. For example, all the data corresponding to the management of partner rewards may be stored in the analytical database 228. The rule-based engine 222 provides defined rules for assignment of rewards to the care partners, the care providers, the wellness partners and other partners that accomplished the tasks as, on target, or if tasks remain unaccomplished, as delayed.

In some embodiments, the recommendation engine 230 may provide a recommendation to the clinical user interface for rewards, corrections, deviations, and management of tasks, targets, patients, care partners, care providers, wellness partners, doctors and disease management partners.

In some embodiments, the recommendation engine 230 may calculate the wellness index of the patient based on one or more factors. For example, the wellness index may be based on patient disease, number of chronic disease, recovery history, medication history, clinical reports and/or some other factors related to the patient.

The care clinical monitoring dashboard module 214 provides a mechanism to the care provider for monitoring the progress by the care providers 218. The care providers 218 can update and/or report the assignments. In some other embodiments, the care providers may use any type of mobile device to update and or report the progress of one or more patients associated with them.

The care clinical monitoring dashboard module 214, the machine learning module 220, and the care providers 218 may be connected to a reward module 232. The rewards module 232 manages the rewards for one or more patients and one or more care network members. In some embodiments, rewards are converted into monetary value and/or non-monetary values by the rewards module 232.

The rewards module 232 is also connected to a care management partner interface 260, a disease management partner interface 270 and a patient device 250 and each of these modules may be connected to each other. The care management partner interface 260 allows the care management team to reward each other when working together as a team to improve the health of the patient.

In some embodiments, the rewards module 232 may also be connected to disease management partner interface 270 to provide capability to review, reward, and provide guidance to the care management team and to correct any deviation in the health improvement from set targets.

The patient device 250 may provide feedback to the partner reward management module 110 regarding the progress, health condition, feelings, mood and at times, self readings on clinical devices of blood pressure, blood sugar, and other such clinical parameters.

This exemplary embodiment of the partner reward management module 110 is just one implementation of the present invention and other variations fall within the scope of the current invention. For example, if a patient has an obesity condition and it is priority #1, then the algorithm may dedicate specific pool of 1000 points to accommodate for 5 care provider network team members including a personal support worker, primary physician, 2 family members and a neighbor. The weightage of the points will be based on the type of tasks being undertaken by each member of the team. In this example scenario, the neighbor, who may be doing most of the tasks for motivation, keeping patient company, helping them with their exercise etc. may be assigned 400 points, the primary physician with very limited interaction and tasks may be assigned 100 points, the personal support worker may be assigned 200 points and each family member may be provided with 150 points each. The metrics may be collected for this patient in regards to their On-Track compliance (responding to reminders for taking meds, exercise reminders, nutrition reminders, questionnaires reminders etc.) and the On-Target compliance (having their weight measured in the optimal range on a weekly basis, having their exercise steps count measured in the optimal range on a weekly basis, taking their meds daily as suggested etc.). These metrics may indicate whether the patient's health condition for obesity is improving or not. Each patient may be assigned a score card with all their tasks and metrics etc. Over time, the deep learning algorithm may start determining how to re-balance the weightage of the points allocation depending on which tasks were most optimal in improving patient's condition and providing positive health outcome for obesity over time. Each member of the network team may be assigned a score card with their tasks, points, weightage of the points etc.

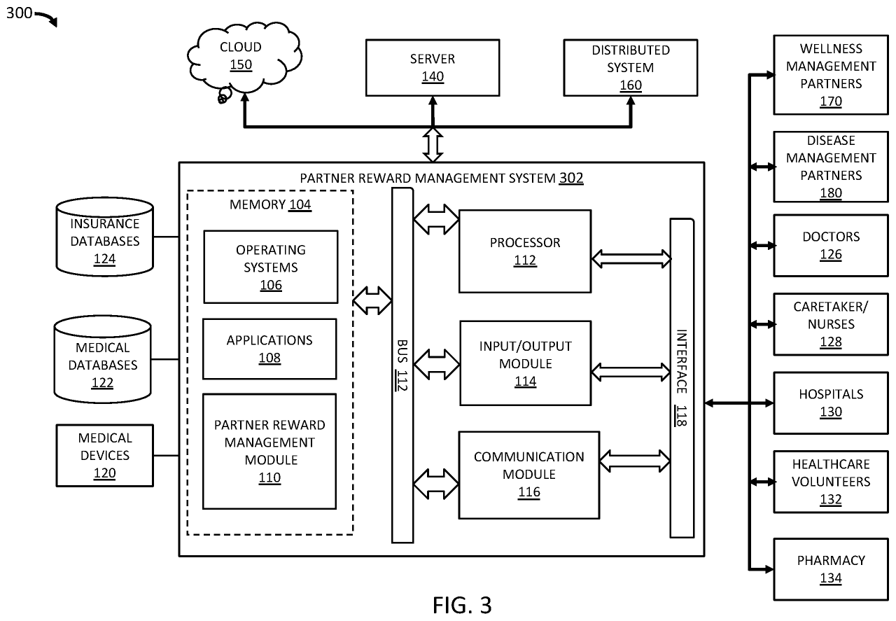

FIG. 3 illustrates a partner reward management system in another embodiment of the present invention. The partner reward management system 302 may be a standalone system for managing the partner rewards or care providers rewards. In some embodiments, the partner reward management system may be a dedicated server which centrally controls all the processes, activities, tasks and manages the partner rewards.

The partner reward management system 302 comprises the memory 104 comprising the operating system 106, the one or more applications 108, and the partner reward management module 110 interfaced with the internal bus 111 to the processor 112, the one or more input/output devices 114, and the one or more communication devices 116. The internal bus 111 carries data and supplies electrical current among different modules. In addition, the partner reward management system 302 includes the interface 118, which allows the partner reward management system 302 to connect with the cloud 150, the server 140 and the distributed system 160. In addition, the medical database 122, the insurance database 124, the medical device 120 may be accessed by the partner reward management system 302 through the internal bus 111 and the interface 118.

The partner reward management system 302 may connect with the one or more wellness management partners 170, the one or more disease management partners 180, the one or more doctors 126, the one or more care takers/nurses 128, the one or more health care volunteers 132, the one or more hospitals 130, and the one or more pharmacy 134. The descriptions for functioning other partner rewards management system is similar to the description provided for the overall environment described in FIG. 1.

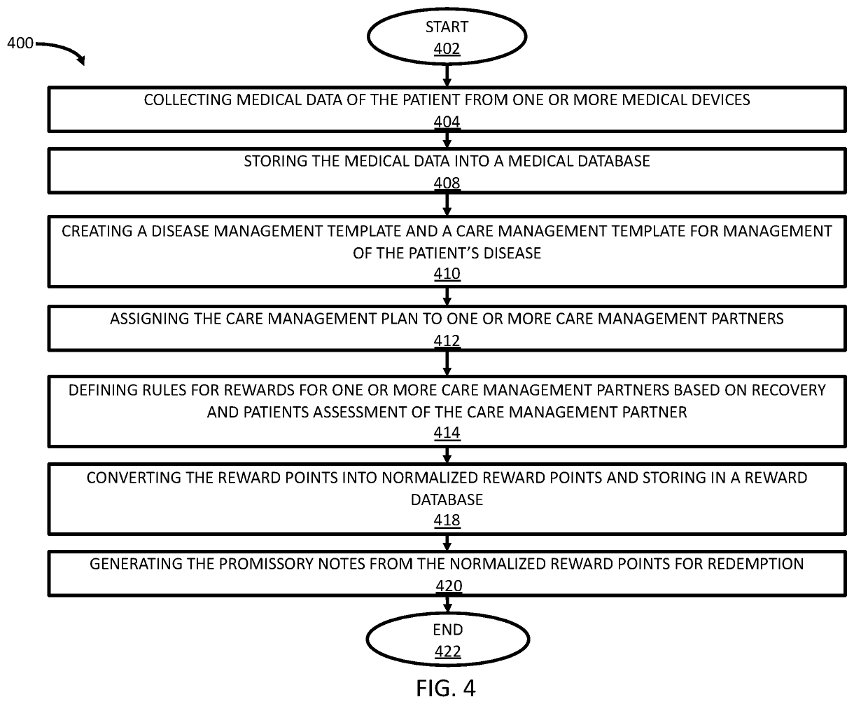

Referring to FIG. 4, the process of partner rewards is illustrated in an embodiment of the present invention. The process starts at 402 by registering the patient with the partner reward management system. At step 404, the patient medical history may be collected from multiple sources such as but not limited to patient database, hospitals, pharmacies, doctors and other medical sources such as clinical laboratories, patient devices, and other sources. The patient may also initiate a process of entering the medical record related to disease(s) from which the patient is suffering. Once the patient enters the history and the other relevant medical data has been collected, the step 408 stores all information related to the patient in the medical database.

At step 410, the process 400 creates a disease template. The disease template may be pre-existing in the system or may be created based on the patient disease(s) and/or requirements. In some embodiments, a group of medical experts may create the disease template rather than one medical expert if the patient has multiple diseases. Alternatively, the patient may request for a specific medical expert based on patient's past experience. At step 412, the process 400 may develop a care management plan from the disease management template as suggested by the one or more medical experts. In some embodiments, the care management plan may be developed by the care partner management interface. In some other embodiments, the care management plan may be developed by one or more medical experts. In some other embodiments, the care management plan may be developed by both disease management partner and a care management partner. In yet another embodiment, the care management plan may be developed through discussions with the patient before date being passed for implementation by a care management partner.

The process 400 may then at step 412 assign the care management plan to one or more care management partners, which may include care providers, wellness partners, family members, health volunteers and others. In some embodiments, the care management plan may be developed by the medical experts and/or care management partners, which may be passed on to the machine learning algorithm and the machine learning algorithm train itself to automate the process of creating care management plan. In this embodiment, the care management plan may be developed using machine learning algorithms based on rules stored in the analytics database.

At step 414, the process 400 may monitor and manage the rewards to be awarded to each of the care partners in the service of the patient based on the compliance of the treatment protocol. In some embodiments, the machine learning algorithm may define the rules for rewards for care management partner based on real time progress in the condition of the patient.

At step 418, the process 400 may convert the accumulated points into normalized reward points. The normalization process may be a formula of converting the reward points into actual redeemable points. The normalized points may be adjusted based on actual contribution of the care provider and/or the achievement of the group of care providers. In sonic embodiments, all of the care providers and the members of a disease management team may be rewarded based on their contribution.

At step 420, the process 400 may generate a coupon or promissory note based on the normalized reward points, which may be redeemed for goods and/or services as specified by the partner reward management system. Once the reward points have been redeemed, the account of the recipient may be adjusted accordingly, or reset to zero or a default value as specified by the system. The process 400 gets terminated at step 422.

A method of rewards for one or more care management partners and one or more disease management partners associated with a patient having one or more ailments, the method comprising: creating a treatment plan for the one or more ailments of the patient; developing a care management plan for the patient based on the treatment plan; assigning the care management plan to the one or a ore care management partners and the one or a ore disease management partners, wherein the rewards are assigned based on one or more health parameters; assigning the rewards to one or more care management partners based on rules related to improvement in the health of the patient; monitoring one or more health parameters associated with the patient, which are managed by one or more care management partners; providing rewards to one or more care management partners and one or more disease management partners based on improvement in the health of the patient.

A reward management system for one or more care management partners and one or more disease management partners associated with a patient having one or more ailments, the reward management system comprising: a data collection module configured to a medical database having information related to the one or more alignments of the patient; a disease template module configured to the medical database and one or more disease management partners for creating a disease template and to provide a treatment plan; a care template module configured to the one or more disease management partners for creating a care management plan of the patient based on the treatment plan; a clinical controller and scheduler module for assigning the care management plan to the one or more care management partners and the one or more disease management partners; a machine learning module for assigning the rewards the one or more care management partners and the disease management partner based on rules related to improvement in the health of the patient; a care clinical monitoring dashboard module for monitoring the one or more health parameters associated with the patient, which are management by the one or more care management partners, and a reward module for providing rewards to one or more care management partners and the one or more disease management partners based on improvement of health of the patient.

In embodiments, the treatment plan may he created based on a disease management template designed by one or more disease management experts.

In embodiments, one or more disease management experts may be associated with one or more disease management partners having research and management experience in treatment of the disease.

In embodiments, one or more care management partners monitor at least one health parameter of the patient

In embodiments, one or more care management partners associated with the treatment plan of the patient ensure treatment plan compliance of the patient.

In embodiments, the rewards are converted into normalized reward points, which may be converted into promissory notes and can be redeemed at certain point of sale or merchant sites.

In embodiments, the normalized reward points are automatically determined by a machine learning algorithm.

In embodiments, the rewards are determined by machine learning algorithms based on health parameters of the patient. In other embodiments the health parameters of the patient may comprise a treatment plan.

In embodiments, the health parameters of the patient comprise clinical text reports. In embodiments, the health parameters of the patient comprise one or more ailments.

We claim:

1. A method of rewards for a care management partner and a disease management partner associated with a patient having an ailment, the method comprising:
creating a treatment plan for the ailment of the patient;
developing a care management plan for the patient based on the treatment plan;
assigning the care management plan to the care management partner and the disease management partner, wherein a reward is assigned based on a health parameter;
assigning the reward to the care management partner based on a set of rules related to an improvement in health of the patient;
monitoring the health parameter associated with the patient;
providing rewards to the care management partner and the disease management partner based on the improvement in health of the patient.

2. The method of claim 1, wherein the treatment plan is created based on a disease management template designed by a disease management expert.

3. The method of claim 1, wherein the care management partner assigned with the care management plan of the patient monitor the health parameter.

4. The method of claim 1, wherein the care management partner assigned with the care management plan of the patient ensures the care management plan compliance of the patient.

5. The method of claim 1, wherein the reward is converted into a normalized reward point, which can be converted into a promissory note which can be redeemed at a point of sale.

6. The method of claim 1, wherein the reward is determined by a first machine learning algorithm based on the health parameter.

7. The method of claim 1, wherein the health parameter of the patient comprises a clinical text report.

8. The method of claim 1, wherein the health parameter of the patient comprises an ailment.

9. The method of claim 2, wherein the disease management expert is associated with the disease management partner having a research and management experience in treatment of the disease.

10. The method of claim 5, wherein the normalized reward point is automatically determined. by a second machine learning algorithm.

11. A partner reward management system for a care management partner and a disease management partner associated with a patient having an ailment, the reward management system comprising:
a data collection module configured to a medical database having information related to the ailment of the patient;
a disease template module configured to the medical database and the disease management partner to create a disease template and to provide a treatment plan;
a care template module configured to the disease management partner to create a care management plan of the patient based on the provided treatment plan;
a clinical controller and scheduler module to assign the care management plan to the care management partner and the disease management partner;
a machine learning module to assign a reward to the care management partner and the disease management partner based on a set of rules related to an improvement in the health of the patient;
a care clinical monitoring dashboard module to monitor a health parameter of the patient; and
a reward module to provide reward to the care management partner and the disease management partner based on the improvement in health of the patient.

12. The partner reward management system of claim 11, wherein the treatment plan is created based on a disease management template designed by a disease management expert.

13. The partner reward management system of claim 11, wherein the care management partner monitors the health parameter of the patient.

14. The partner reward management system of claim 11, wherein the care management partner assigned the care management plan of the patient ensures care management plan compliance of the patient.

15. The partner reward management system of claim 11, wherein the reward is converted into normalized reward point, which can be converted into a promissory note which can be redeemed at a point of sale.

16. The partner reward management system of claim 11, wherein the reward is determined by a first machine learning algorithm based on the health parameter of the patient.

17. The partner reward management system of claim 11, wherein the health parameter of the patient comprises a clinical text report.

18. The partner reward management system of claim 11, wherein the health parameter of the patient comprises the ailment.

19. The partner reward management system of claim 12, wherein the disease management expert is associated with the disease management partner having research and management experience in treatment of the disease.

20. The partner reward management system of claim 15, wherein the normalized reward point is automatically determined by a second machine learning algorithm.
